(19) United States
(12) Patent Application Publication
(10) Pub. No.: US 2006/0088850 A1
(43) Pub. Date: Apr. 27, 2006

(54) Method for genome-wide analysis of palindrome formation and uses thereof

(75) Inventors: Stephen J. Tapscott, Seattle, WA (US); Hisashi Tanaka, Seattle, WA (US); Meng-chao Yao, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fred Hutchinson Cancer Research Center, Seattle, WA (US)

(21) Appl. No.: 11/142,091

(22) Filed: May 31, 2005

Related U.S. Application Data
(60) Provisional application No. 60/575,331, filed on May 28, 2004.

(51) Int. Cl. C12Q 1/68

(52) U.S. Cl. 435/6

(57) ABSTRACT

The present invention provides a method for rapidly detecting the genome-wide presence of palindrome formation. The method has demonstrated that somatic palindromes occur frequently and are widespread in human cancers. Individual tumor types have a characteristic non-random distribution of palindromes in their genome and a small subset of the palindromic loci are associate with gene amplification. The disclosed method can be used to define the plurality of genomic DNA palindromes associated with various tumor types and can provide methods for the classification of tumors, and the diagnosis, early detection of cancer as well as the monitoring of disease recurrence and assessment of residual disease.

### CROSS-REFERENCES TO RELATED APPLICATIONS

The present application claims priority to U.S. Provisional Patent Application No. 60/575,331, filed May 28, 2004, the entire disclosure of which is incorporated by reference herein.

### STATEMENT AS TO RIGHTS TO INVENTIONS MADE UNDER FEDERALLY SPONSORED RESEARCH AND DEVELOPMENT

Aspects of the present invention were conducted with funding provided by the National Institutes of Health under Grant Nos. R01AR 045113 and R01GM 26210. The Government may have certain to rights in the claimed invention.

### BACKGROUND OF THE INVENTION

Cancer is a disease of impaired genetic integrity. In most cases disturbed genetic integrity is observed at the chromosome level and include a configuration called anaphase bridges, which are most likely derived from dicentric or ring chromosomes segregating into two different daughter cells in the process of the breakage-fusion-bridge (BFB) cycle. The BFB cycles have been shown to generate large DNA palindromes with structural gains and losses at the termini of sister chromatids by creating recombinogenic free ends, followed by sister chromatid fusions at each cycle. Evidence has been accumulating that the BFB cycle is a major driving force for genetic diversity generating chromosome aberrations in cancer cells. Telomere shortening in mice lacking the Telomerase RNA component (TR) results in chromosome end-to-end fusions that are enhanced by p53 deficiency. Initiation of neoplastic lesions and frequent anaphase bridges are both increased with progressive telomere shortening in mouse intestinal tumors, and human colon carcinomas show a sharp increase of anaphase bridges at the early stage of carcinogenesis. This suggests that telomere dysfunction can generate dicentric chromosomes by end-to-end fusions and trigger the BFB cycle, providing genetic heterogeneity that furthers the malignant phenotype. Spontaneous and/or ionizing radiation induced chromosome end-to-end fusions are also seen in cells that have cancer-predisposing mutations, such as a deficiency in the DNA damage checkpoint function (ATM) (Metcalf et al. Nat. Genet. 13:350-353 (1996)), non-homologous end-to-end joining (NHEJ) repair of DNA double strand breaks (DSB) (DNA-PKcs, Ku70, Ku80, Lig4, XRCC4) (Bailey et al., Proc. Natl. Acad. Sci. USA 96:14899-14904 (1999); Ferguson et al., Proc. Natl. Acad. Sci. USA 97: 6630-6633 (2000); Gao et al., Nature 404:897-900 (2000); Hsu et al., Genes Dev. 14:2807-2812 (2000)), RAD51D (Tarsounas et al., Cell 117:337-347 (2004)) and histone H2AX (Bassing et al., Proc. Natl. Acad. Sci. USA 99:8173-8178 (2002)). Moreover in mice deficient in both p53 and NHEJ, co-amplification of c-myc and IgH in pro B cell lymphomas is initiated by the BFB cycle after RAG-induced DSB at the IgH locus is incorrectly repaired by fusion to the c-myc gene to form a dicentric chromosome (Gao et al., supra. (2000); Zhu et al., Cell 109: 811-821 (2002)). This indicates that improper DSB repair also could trigger the BFB cycle for further chromosome aberrations.

The BFB cycle has also been implicated as a common mechanism for intrachromosomal gene amplification (Coquelle et al., Cell 89:215-225 (1997); Ma et al., Genes Dev. 7:605-620 (1993); Smith et al., Proc. Natl. Acad. Sci. USA 89:5427-5431 (1992); Toledo et al., EMBO J. 11:2665-2673 (1992)). Studies of gene amplifications selected by drug resistance in rodent cells have shown that most of the amplifications are associated with large DNA palindromes (Coquelle et al., supra. (1997); Ma et al., supra. (1993); Ruiz and Wahl, Mol. Cell Biol. 8:4302-4313 (1988); Smith et al., Proc. Natl. Acad. Sci. USA 89:5427-5431 (1992); Toledo et al., supra. (1992)). An initial palindromic duplication of the dhfr gene induced by I-SceI-induced chromosomal DSB triggers BFB cycles and results in further dhfr amplification, where the initial formation of a palindrome appears to be the rate-limiting step for subsequent gene amplification (Tanaka et al., Proc. Natl. Acad. Sci. USA 99:8772-8777 (2002)). Various clastogenic drugs induce initial chromosome breaks at the common loci that bracket the palindromic amplification of the selected gene (Coquelle et al., supra. (1997)), suggesting the presence of specific loci in the genome susceptible to palindrome formation.

Although cytogenetic studies of cancer cells also indicate that oncogene amplifications occur as large DNA palindromes by BFB cycles (Ciullo et al., Hum. Mol. Genet. 11:2887-2894 (2002); Hellman et al., Cancer Cell 1:89-97 (2002)), little is known about how prevalent this type of chromosome aberration is in cancer cells. Given the fact that telomere dysfunction and impaired DNA damage checkpoint/repair functions can trigger BFB cycles and are major causes of chromosome instability, somatic palindrome formation might be widespread in cancer cells and provide a platform for additional gene amplification. However, our molecular analysis of the structure of amplified loci in cancer cells has been limited by the fact that the duplication covers very large regions of the chromosome.

DNA methylation in vertebrates is a well-established epigenetic mechanism that controls a variety of important developmental functions including X chromosome inactivation, genomic imprinting and transcriptional regulation. Cytosine DNA methylation in mammals predominantly occurs at CpG dinucleotides, of which more than 70% are methylated. CpG islands are clusters of CpG dinucleotides that mostly remain unmethylated and could play an important role in gene regulation. There are approximately 27,000 and 15,500 CpG islands in the human and mouse genomes respectively, among which 10,000 are highly conserved between these two organisms. CpG islands often reside in 5′ regulatory regions and exons of genes (promoter CpG islands), and recent computational analysis indicates that a significant proportion of CpG islands are in other exons and intergenic regions. Although CpG islands are generally considered to be unmethlylated, a significant fraction of them can be methylated. For example, a number of studies have shown that differential methylation of promoter CpG islands leads to transcriptional repression of tumor suppressor genes in cancer cells. There also are a few CpG islands that undergo tissue specific methylation during development. However, these examples are limited in number and fail to reveal the full scope of dynamic changes in methylation status. For instance, there is general hypomethylation in cancer cells, and a genome-wide demethylation-remethylation transition occurs during normal development. For evaluation of genome-wide DNA methylation of CpG islands, it may be necessary to develop a robust microarray-based method.

The present invention provides a rapid method for the study of the genome-wide distribution of somatic palindrome formation. In particular, the method provides a procedure to identify chromosomal regions susceptible to subsequent gene amplification associated with cancer and other conditions. This method can serve as a sensitive technique to detect early stages of tumorigenesis since in many cases chromosome aberration are early manifestations of malignant transformation. The method has also be adapted to amplify DNA enriched for unmethylated CpG islands.

### BRIEF SUMMARY OF THE INVENTION

A genome-wide method for identifying a region of genomic DNA comprising a DNA palindrome is disclosed. The method generally comprises incubating isolated fragmented total genomic DNA under conditions conducive to snap back DNA formation and not inter-molecular hybridization, the snap back DNA containing the DNA palindrome; isolating the snap back DNA; and identifying the regions of the genomic DNA comprising the snap back DNA to identify those regions of the genomic DNA comprising the DNA palindrome. In a more particular embodiment the method comprises fragmenting the total genomic DNA with, for example a restriction enzyme, denaturing the genomic DNA, incubating the fragmented, denatured genomic DNA under conditions conducive to the formation of snap back DNA in those regions of the DNA comprising the DNA palindrome; and identifying the region of the genomic DNA containing the DNA palindrome by hybridization with an array comprising human genomic DNA.

In a preferred embodiment, the method comprises the steps of: a) isolating genomic DNA comprising the DNA palindrome from a population of cells; b) denaturing the isolated DNA; c) rehybridizing the denatured isolated DNA under suitable conditions for the DNA palindrome to form snap back DNA; d) digesting the rehybridized DNA with a nuclease that digests single stand DNA to form double stranded DNA fragments comprising the snap back DNA; e) digesting the double stranded DNA fragments comprising the snap back DNA with a nucleotide sequence specific restriction enzyme; f) adding a sequence specific linker nucleotide sequence to one end of each stand of the double stand DNA comprising the snap back DNA; g) amplifying the DNA fragments comprising the added linker using a labeled linker sequence specific primer corresponding to the sequence specific linker added in step (f); and h) hybridizing the amplified DNA fragments comprising the snap back DNA to a genomic DNA library and identifying the genomic DNA region comprising the palindrome.

The method can further comprise the step of mixing and co-hybridizing the amplified DNA fragments comprising the snap back DNA with a sample of high molecular weight total genomic DNA fragments that has not been incubated to form snap back DNA. As with the snap back DNA sample, the normal high molecular weight DNA will have been digested with S1 nuclease and with the same restriction enzymes of step (e) as the snap back DNA sample, have the sequence specific linker added and the DNA fragments amplified and labeled using a sequence-specific primer corresponding to the sequence specific linker added in the previous step which contains a second label, prior to mixing with the snap back DNA and co-hybridization.

Any single strand nuclease can be used in the present method including, for example S1 nuclease. Further, as well known in the art the genomic DNA fragments can be digested with any restriction enzyme that specifically cuts double stranded DNA. Typically, the DNA will be digested with two or more restriction enzymes and the profiles compared. In one embodiment of the present invention the DNA is digested separately with MspI, TaqI, or MseI. To prepare the high molecular weight genomic DNA, total DNA from a sample of a cell population is isolated by methods well know to the skilled artisan and the isolated genomic DNA is fragmented by a chemical, physical, or enzymatic method. In one embodiment the genomic DNA is digested with, for example, SalI, but any other restriction enzyme that results in high molecular weight DNA can also be used.

The present invention also provides a method for classifying a population of cancer cells. The method comprises identifying a plurality of snap back DNA regions that contain a palindrome and using the identity of the plurality of genomic DNA regions each comprising the palindromes to classify the population of cancer cells. Typically, the method comprises fragmenting the genomic DNA; denaturing the genomic DNA; incubating the fragmented, denatured genomic DNA under conditions conducive to the formation of snap back DNA by regions of the genomic DNA comprising the DNA palindrome; and identifying the plurality of regions of the genomic DNA containing the DNA palindrome to form a profile unique to the population of cells. The method can further comprise comparing the profile of genomic DNA comprising a palindrome of the cancer cell population to a population of normal cells or to a profile established for another tumor type.

A method for detecting a population of cancer cells, comprising isolating genomic DNA from a cell population, identifying a plurality of snap back DNA regions that comprise genomic DNA regions containing a palindrome and using the identity of the plurality of genomic DNA regions comprising the palindromes to detect the population of cancer cells. More specifically, the method comprises fragmenting the genomic DNA to form high molecular weight fragments; denaturing the fragmented genomic DNA; incubating the fragmented, denatured genomic DNA under conditions conducive to the formation of snap back DNA by regions of the DNA comprising the DNA palindrome, the conditions not being conducive to forming inter-molecular bonds; and identifying the region of the genomic DNA containing the DNA palindrome to form the profile. The method can further comprise comparing the palindrome profile of the cancer cell population to a population of normal cells or to a palindrome profile of another tumor cell population.

### BRIEF DESCRIPTION OF THE DRAWINGS

FIGS. 1A through C provide results of a series of experiments with a cell line comprising a large palindrome of the DHFR transgene (D79IR-8 Sce2 cells, WO 03/029438, incorporated herein by reference) demonstrating that the genome-wide assessment of palindrome formation assay efficiently generate intra-molecular base pairings in large palindromic sequences (‘snap-back’ DNA or SB DNA) and that these can be used to isolate large palindromic fragments from total genomic DNA. FIG. 1A depicts the NaCl-dependent formation of ‘snap-back’ (SB) DNA. Genomic DNA obtained from the CHO DHFR-cells containing inverted duplication of the DHFR transgene was heat denatured and rapidly cooled on ice. KpnI or XbaI digestion of DNA and Southern blotting demonstrated efficient intra-strand hybridization of the duplicated region. A 5 kb fragment of KpnI digest and an 11 kb fragment of XbaI digest, respectively, each of which is the size expected for the snap back DNA, were seen on the Southern blot in a NaCl-dependent manner. Solid lines and dotted lines represent single stranded DNA that was complimentary to each other. Probe used for hybridization is indicated on the figure. FIG. 1B depicts the same genomic DNA from D79IR-8 Sce2 cells as in FIG. 1A which was digested with SalI. The SalI-digested DNA was denatured, renatured, and subjected to S1 digestion. The couble-stranded DNA was then digested with MspI or TaqI and the digested DNA was amplified by ligation-mediated PCR using linker specific primers. The DNA products were analyzed by Southern blot with a probe for a fragment that contains an inverted repeat (Probe 1), or a probe to an adjacent region that did not contain an inverted repeat (Probe 2). Signals were detected exclusively with the probe to the fragment with the inverted repeat (Probe 1), indicating that DNA obtained by this method is highly enriched for genomic sequences with palindromes. FIG. 1C examines whether the measurement of somatic palindromes could minimize the effect of non-palindromic counterpart. SalI-digested genomic DNA from D79IR-8 Sce2 and parental cells were mixed in a variety of ratios such that the total amount of DNA was 4 μg Two micrograms of DNA were subjected to snap back and amplification by LM-PCR for PCR-Southern analysis (upper panel), and the remaining 2 μg of the mixed DNA was digested with KpnI and analyzed by genomic Southern (lower panel). Both Southern analyses were hybridized with a probe specific for inverted repeat (Probe 1 from FIG. 1B). Unlike the signals on the genomic Southern blot, specific signals from the palindrome were seen even after 1/40 dilution, indicating that this approach can detect somatic palindrome formation in a subpopulation of cells.

FIG. 2 is a pictorial summary of the “Procedure of Genome-wide analysis of Palindrome Formation” (GAPF). Tumor samples were subjected to the process to produce snap back DNA, treated with single strand specific nuclease S1, digested with either MspI, TaqI or MseI, ligated with a specific linker having the appropriate complementary sequence (MspI, TaqI or MseI), and amplified by PCR with Cy5-labeled linker specific primer. Standard DNA was prepared from normal human fibroblast (HFF) DNA by the same method except for the snap back process, and labeled with Cy3. Labeled DNAs were co-hybridized onto a human spotted cDNA microarray.

FIG. 3 depicts various comparisons of GAPF features between normal human fibroblasts, normal breast epithelial cells, epithelial cancer cell lines, and the pediatric cancers medulloblastoma and rhabdomyosarcoma. FIG. 3A compares the features of three normal human fibroblast preparations. No significant difference in GAPF features between normal human fibroblasts were observed. Features of SB-DNA of three independent primary cultures of fibroblasts (HDF1 (skin biopsy), HFF2 (foreskin sample) and HFF3 (skin biopsy)) were compared with non-SB-DNA of HFF2 as the common standard, genomic DNA of HFF2 without denaturation and renaturation (non-SB-DNA). Experiments were carried out in triplicate for each set of hybridization using three different preparations of templates. For each gene in each comparison, the q-value, which is a measure of significance in terms of false discovery rate (FDR), was calculated. In these analyses, thresholding genes with q-value<0.1 calls no genes significantly different between any two normal fibroblasts samples. The values pi(0), which represents the percentage of true negatives, and the minimum q-value (qₘᵢₙ) indicate that two sets of SB-DNA (HDF1 and HDF3) are almost identical, while that of HFF2 was very closely related to those of HDF1 and HDF3. FIG. 3B examines cancer specific somatic palindrome formations. GAPF features from HFF2 (normal human foreskin fibroblast, three independent hybridizations on microarrays, N=3), AG32 (normal breast epithelial cell line, N=3), HDF3 (normal human fibroblast, independent from FIG. 3A, N=5), Colo320DM (colon cancer cell line, N=3), MCF7 (breast cancer cell line, N=3), RD (rhabdomyosarcoma cell line, N=3) and five independent medulloblastoma tissues were compared to a common baseline profile consisting of two triplicate data sets of SB-DNA from HDF1 and HDF3 (FIG. 3A). The data from individual genes was grouped into 521 cytogenetic bands, and bands with q<0.05 and log(fold change)>0 were called ‘significantly increased’ relative to the common baseline. Numbers between each cell line and common baseline represent the number of significantly increased cytogenetic bands relative to the common baseline in the cell line. FIG. 3C examines the overlaps in areas of palindrome formation. Significant overlaps of somatic palindrome containing bands were found among age-related epithelial cancers (Colo320DM and MCF7, p=4.4427×10⁻⁶) or pediatric cancers (medulloblastomas and RD, p=0.017). FIG. 3D examines the distribution of overlaps of palindrome containing cytogenetic bands between age-related epithelial cancers and pediatric cancers. Neither Colo320DM nor MCF7 showed significant overlap of palindrome-containing cytogenetic bands with those of medulloblastoma or RD.

FIGS. 4A through 4C depict the clustering of somatic palindromes at specific regions of the genome in Colo320DM and MCF7. Genes form each loci and the surrounding region were plotted on the physical map and fold change of the GAPF and CGH (comparative genomic hybridization) features relative to HDF and are shown. Arrows indicate significant increases (q<0.05) either in Colo (black) or MCF7 (grey). FIG. 4A depicts the profiles of a 32 mega-base regions of the long arm of chromosome 8. The somatic palindromes commonly clustered in two regions at 8q24.1. Palindromes commonly cluster at the MYC gene and 5 MB centromeric to MYC. Note that palindrome formation was associated with the copy number increase of MYC, but not the genes at 5 MB centromeric in Colo320DM. FIG. 4B depicts the profiles of the 18 MB region at 1q21 and a detailed profile of the 4 MB clustered region. The data demonstrate a common cluster of somatic palindromes at a 600 kb region at 1q21. FIG. 4C depicts the palindrome profile of the region corresponding to the common fragile site Fra7I at 7q35.

FIGS. 5A and 5B depict a comparison of the snap back DNA profiles for a human foreskin fibroblast cell population and the human colon cancer cell line Colo320DN. FIG. 5A. The human colon cancer cell line Colo320DM contains an inverted duplication of the c-myc gene. Left panel; Southern blotting analysis of genomic DNA from either Colo320DM or human foreskin fibroblast (HFF). DNA rearrangement is seen in the Colo320DM. Denaturation and rapid renaturation (snap back, SB) of HFF DNA shows loss of the EcoRI fragment. Right panel; Genomic DNA from Colo320DM was either: (a) digested with EcoRI and then subjected to snap-back (EcoRI→SB); or, (b) subjected to snap-back and then digested with EcoRI (SB→EcoRI). Digesting with EcoRI prior to snap-back disrupts the inverted repeat following denaturation and results in fragments that will remain single stranded following snap-back and will be sensitive to S1 nuclease. In contrast, when snap-back is performed prior to EcoRI digestion, the intact inverted repeat will efficiently form double stranded DNA through intra-strand pairing, producing S1 nuclease resistant fragments following EcoRI digestion. Southern hybridization was done using a human c-myc cDNA probe. FIG. 5B. The ECM1 gene was amplified as an inverted repeat and was subjected to snap back. Southern analysis of SB-DNA from Colo320DM shows a half-size EcoRI fragment relative to that of non-SB-DNA, indicating a palindromic amplification of ECM1. Right panel; A human myogenin probe was cohybridized as a control. Left panel; no fragment was seen on the SB-DNA from Colo320DM DNA by hybridizing with the myogenin probe only.

FIG. 6 depicts the hierarchical clustering of the GAPF profile of 5 medulloblastomas and three normal fibroblasts (HDF3). A high degree of similarity among five individual medulloblastomas was seen, which is clearly separable from normal fibroblasts.

FIG. 7 is an idiogram showing genome wide distribution of somatic palindromes. Palindrome-containing cytogenetic bands are shown on the right side of chromosome (Colo320DM, left column of circles, and MCF7, right column of circles) or on the left side (medulloblastoma, right column of circles, or RD, left column of circles). The cytogenetic bands with palindromes that are identified in both Colo and MCF7 cluster at 1q21, 8q24.1, 12q24, 16p12-13.1 and 19q13.

FIGS. 8A and 8B provide a schematic and data for using ligand-mediated methylation PCR to amplify DNA fragments enriched for unmethylated CpG islands. FIG. 8A provides a schematic for the process of ligand-mediated methylation PCR for amplification of unmethylated CpG islands. FIG. 8B provides a blot showing the amplification of small (<500 base pair) HpaII DNA fragments.

### DETAILED DESCRIPTION OF THE INVENTION

Generally, the nomenclature used herein and many of the laboratory procedures in regard to cell culture, molecular genetics and nucleic acid chemistry and hybridization, which are described below, are those well known and commonly employed in the art. (See generally Sambrook et al., Molecular Cloning: A Laboratory Manual, 3d Ed., Cold Spring Harbor Laboratory Press, New York (2001), which is incorporated by reference herein). Standard techniques are used for recombinant nucleic acid methods, preparation of biological samples, preparation of cDNA fragments, PCR, and the like. Generally enzymatic reactions and any purification and separation steps using a commercially prepared product are performed according to the manufacturers' specifications. Although specific enzymes and other recombinant nucleic acid methods and products are described and used, other enzymes and recombinant nucleic acid methods and products are well known in the art and are available for use in the described methods.

Loss of chromosome integrity in human cancers generates numerous gains and losses of chromosome segments. Large DNA palindromes caused by Breakage-Fusion-Bridge (BFB) cycles might facilitate gene amplification in human cancers, however, the prevalence of initial palindrome formation is largely unknown. In the present invention a novel microarray-based approach called Genome-wide Analysis of Palindrome Formations (GAPF) is used to demonstrate that somatic palindrome formation is widespread and non-random in human cancers. Individual tumor types appear to have a characteristic distribution of palindromes in their genome and only a subset of these palindromic loci are associated with gene amplification. The present disclosure identifies widespread palindrome formation in human cancer that can provide a platform for subsequent gene amplification and indicates that tumor specific mechanisms determine the locations of palindrome formation. A method for rapidly identifying the genomic DNA locations of palindrome formation in various populations of cells in provided herein, as well as applications of the methods for characterizing tumor types, palindrome regions susceptible to gene application and their association with cancer diagnosis and early cancer detection, assessment of residual disease, and monitoring for disease recurrence.

Provided herein is a novel microarray based approach designated Genome-wide Analysis of Palindrome Formation (GAPF). By using this approach it has been found that somatic palindrome formation is in fact a common form of chromosome instability and that these palindrome formations tend to cluster at specific loci in the genome, “hotspots for palindrome formation.” Surprisingly, use of the method disclosed herein has revealed that individual tumor types appear to have a characteristic distribution of palindromes in their genome, indicating that tumor specific mechanisms determine the locations of palindrome formation. Somatic palindromes are not always associated with significant gene amplification, whereas loci with high-level amplifications are usually accompanied by somatic palindromes. These data indicate that the somatic formation of palindromes broadly alters the cancer genome and provides a platform for subsequent gene amplification.

Ligation-mediated PCR (LM-PCR) can also be used to amplify DNA enriched for unmethylated CpG islands. The method can be used, for example, to study differential methylation between cancer and normal cells, and tissue specific methylation during differentiation. The method generally can use genomic DNA from any cell population, tissue sample, and the like. The cell population or tissue samples that can be used in the method include any normal tissue, such as skin, blood, bladder, lung, prostate, brain, ovary, and the like, a tumor, such as a melanoma, leukemia, bladder tumor, lung tumor, prostate tumor, brain tumor, ovarian tumor, and the like, or any other tissue or organ at a particular point in development. Genomic DNA from a cell population or tissue sample is digested with a methylation sensitive restriction enzyme. Methylation sensitive restriction enzymes useful in the present invention include, for example, HpaII, and the like. Prior to digestion the genomic DNA can be fragmented by known physical, chemical or enzymatic means to form high molecular weight DNA. The high molecular weight DNA can then be further digested with the methylation sensitive restriction enzyme.

### EXAMPLES

### Example 1

The following example describes the process for genome-wide assessment of palindrome formation.

Methods

Cell Lines and Cancer Tissues

D79IR-8 and D79IR-8-Sce 2 cells were previously described (Tanaka et al., Proc. Natl. Acad. Sci. USA 99:8772-8777 (2002)). Colo320DM and RD were obtained from American Type Culture Collection. MCF7 and AG1113215 were from the University of Washington. Skin biopsy derived fibroblasts HDF1 and HDF3 were obtained from the University of Washington and human foreskin fibroblasts HFF2 from the Fred Hutchinson Cancer Research Center (FHCRC) as anonymous cell lines. DNA samples stripped of identifying information from five primary medulloblastomas were provided by the FHCRC. All samples were obtained after FHCRC Institutional Review Board review and approval for use of anonymous human DNA samples and human cell lines.

Linkers and Oligos

Oligonucleotides were synthesized by QIAGEN Genomics. For ligation mediated PCR, two oligonucleotides were annealed in the presence of 100 mM NaCl; for MspI digested DNA, JW102g-5′-GCGGTGACCCGGGAGATCTGAATTG-3′ (SEQ ID NO:1) and JW103pc2-5′-[Phosp]CGCAATTCAGATCTCCCG-3′ (SEQ ID NO:2), for TaqI digested DNA, JW102-5′-GCGGTGACCCGGGAGATCTGAATTC-3′ (SEQ ID NO:3) and JW103p2 5′-[Phosp]CGGAATTCAGATCTCCCG-3′ (SEQ ID NO:4), and for MseI digested DNA, JW102g- and JW103pcTA-5′ -[Phosp]TACAATTCAGATCTCCCG-3′ (SEQ ID NO:5). To label DNA for microarray, the following linker specific primers were end-labeled either with Cy3 or Cy5 and used for PCR; for MspI linker ligated DNA, JW102gMSP-5′-GCGGTGACCCGGGAGATCTGAATTGCGG-3′ (SEQ ID NO:6), for TaqI linker ligated DNA, JW102Taq-5′-GCGGTGACCCGGGAGATCTGAATTCCGA-3′ (SEQ ID NO:7), for MseI linker ligated DNA, JW102gMse-5′-GCGGTGACCCGGGAGATCTGAATTGTAA-3′ (SEQ ID NO:8).

To make a probe for Southern analysis, human genomic DNA was amplified by PCR and a fragment was cloned (TOPO TA Cloning® Kit (Invitrogen)). Oligos used for PCR were; for ECM1, ECM15154, 5′-ACACCTTTCACACCTCGCTTCTC-3′ (SEQ ID NO:9) and ECM15851 5′-GGCAGATAAAGAAGAGACAGTGGTTG-3′ (SEQ ID NO:10).

Microarray Analysis

To make a snap-back DNA, 2 μg of high molecular weight genomic DNA in 50 μl with 100 mM NaCl was boiled for 7 minutes and transferred on ice to cool it down quickly. 6 μl of S1 nuclease buffer, 4 μl of 3 M NaCl and 100 Units of S1 nuclease (Invitrogen) was added to the DNA and incubated at 37° C. for about one hour. S1 nuclease was inactivated by 10 mM EDTA and phenol/chloroform extraction. DNA was precipitated by ethanol and dissolved in water and digested with 40 U of MspI, TaqI or MseI for 16 hours. DNA was precipitated, dissolved into 21 μl of water and ligated to a MspI, TaqI or MseI specific linker by adding 5 μl of 20 mM linker, 3 μl of T4 DNA ligase buffer and 400 U of T4 DNA ligase at 16° C. for about 16 hours. DNA was precipitated and dissolved into 200 μl TE, followed by being applied onto a centrifugal filter unit (MICROCON YM-50; Millipore) to remove an excess of linker. DNA was recovered in 20 μl water. Thus for each cell line or tumor tissue, templates with three different linkers were prepared. For PCR, 2 μl of DNA, 0.5 μl of Taq DNA polymerase (FASTSTART Taq DNA polymerase; Roche), 2.5 μl of 2 mM dNTP, 5 μl of 10×PCR buffer, 2 μM of a Cy3 or Cy5 labeled linker-specific primer were mixed with water to a total of 50 μl reaction. PCR was performed at 96° C. for 6 minutes followed by 30 cycles of 96° C. for 30 sec, 55° C. 30 sec and 72° C. 30 sec on a 9600 Thermal Cycler (Perkin-Elmer). PCR reactions for the same template from different linker specific primer were mixed and purified (PCR purification Kit; QIAGEN). Human Cot-1 DNA (100 μg), poly polydA/dT (20 μg), and yeast tRNA (100 μg) were added for hybridization to a 18 k human cDNA array. For primary medulloblastoma, each tumor sample was processed as a singleton and the GAPF profiles from the five independent samples were compared to the HDF GAPF profile. To prepare template DNA for array-CGH analysis, genomic DNA was digested with MspI, TaqI or MseI, and ligated with a linker specific for each restriction enzyme. Three independent preparation of template DNA were amplified either by Cy3 or Cy5 labeled linker-specific primer. Triplicated co-hybrydization of either Cy3-labeled cancer (Colo320DM or MCF7) DNA with Cy5-labeled normal (HFF2) DNA or Cy5-labeled cancer DNA with Cy3-labeled normal DNA was performed. Oligonucleotides were synthesized by QIAGEN Genomics.

Southern Blotting

Southern blotting was performed as described previously. Briefly, 2 μg of high molecular weight human genomic DNA was digested with restriction enzyme, run on 0.8% agarose gel and blotted to nylon membrane. Snap-back DNA was prepared as follows; 2 μg of genomic DNA in 50 μl water with 100 mM NaCl was boiled for 7 minutes and immediately transferred on ice to be cooled down. DNA was precipitated by ethanol, and digested with restriction enzyme. 2.5 kb Molecular Ruler (BIO-RAD), 1 kb DNA ladder and 100 bp DNA ladder (New England Biolabs) were used as size markers. To make a probe for Southern analysis, human genomic DNA was amplified by PCR and a fragment was cloned by TOPO TA Cloning Kit (Invitrogen). Oligo primer sequences are available on request.

Statistical Analysis

Array data was normalized in the GeneSpring Analysis Package, version 6.2 (Silicon Genetics, Redwood City, Calif.) using Lowess normalization (an intensity-dependent algorithm). The data was then transformed into logarithmic space, base 2. Data was annotated by cytogenetic band or by UniGene cluster using NCBI databases current as of February, 2004. Welch's t-test was performed for each cytogenetic band or UniGene cluster comparing replicate data sets. Storey's q-value was used to control for multiple testing error and each p-value was transformed to a q-value, which is an estimate of the false discovery rate.

Results

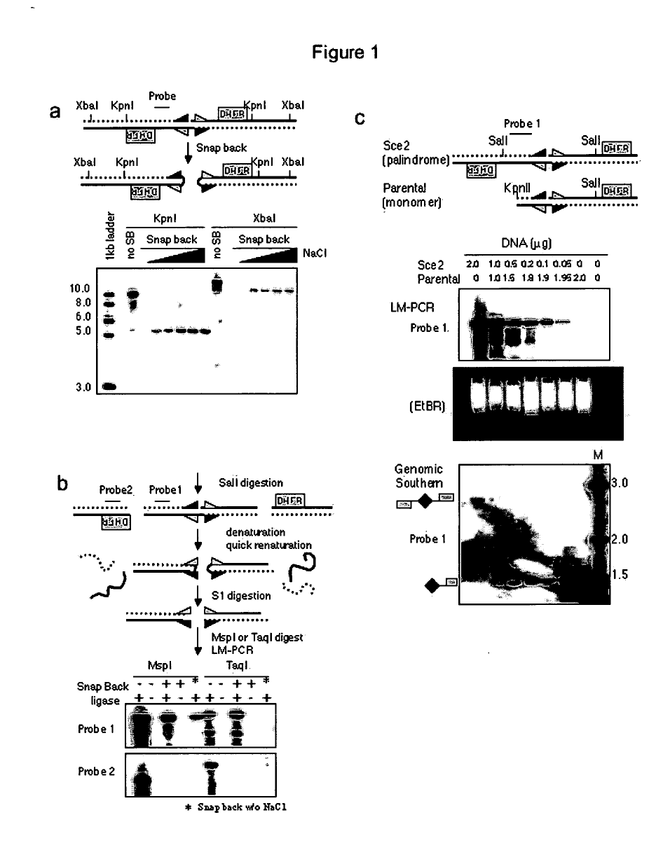

A method to obtain a genome-wide assessment of palindrome formation is disclosed herein based on the efficient generation of intra-molecular base pairing in large palindromic sequences. (Ish-Horowicz et al., J. Mol. boil. 142:231-245 (1980); Ford and Fried, Cell 45:425-430 (2986). Palindromic sequences can rapidly anneal intramolecularly to form “snap-back” (SB) DNA under conditions that do not favor inter-molecular annealing. Snap-back DNA formation can be demonstrated from an endogenous palindrome after heat denaturation and rapid cooling of genomic DNA from cells that contain a few copies of a large palindrome of the DHFR transgene (D79-8 Sce2 cells) (FIG. 1A). The decreased size of the restriction length fragment—the 11 kb KpnI fragment becomes 5.5 kb and the 24 kb XbaI fragment becomes 12 kb, respectively—indicates that renaturation occurs through intramolecular base-pairing.

To determine whether the efficient formation of snap-back DNA could be used to isolate large palindromic sequences from total genomic DNA, genomic DNA from D79-8 Sce2 cells was digested with SalI, followed by denaturation, rapid-renaturation, and digestion with the single strand specific nuclease S1. The snap-back DNA formed by palindromes should be relatively resistant to S1 nuclease, whereas the remainder of the genomic DNA will not efficiently re-anneal and should be S1 sensitive (FIG. 1B). S1 resistant double-stranded DNA was amplified by ligation-mediated (LM) PCR using linker-specific primers after digestion with MspI or TaqI and detected by Southern blotting with either a probe within the inverted repeat (probe 1) or a probe in an adjacent non-palindromic fragment (probe 2). A signal was detected exclusively with the probe to the palindromic fragment, indicating that the genomic DNA obtained by this method was highly enriched for palindromic sequences. This also demonstrated that the enrichment depended on the structure of the DNA, not the copy number of the gene, because the copy number was the same for the fragment with the inverted repeat and the adjacent non-panlindromic fragment.

A dilution experiment was performed to demonstrate that this technique can identify genomic palindromes that exist in a sub-population of cells, such as might occur in a tumor with a heterologous population of genetically altered cells, such as provided by an intratumoral heterogeneity. Genomic DNA from D79IR-8 Sce2 cells was serially diluted with DNA from the parental cells that contained a single non-palindromic copy of the transgene. The DNA mixes were analyzed by standard genomic Southern analysis (FIG. 1C, lower panel) or subjected to snap-back, amplification by LM-PCR, and then Southern analysis (FIG. 1C, upper panel). Using a probe specific to the inverted repeat (probe 1 from FIG. 1B), specific signal from the palindrome was seen even after a 1/40 dilution, demonstrating that this approach can detect a somatic palindrome in a sub-population of cells.

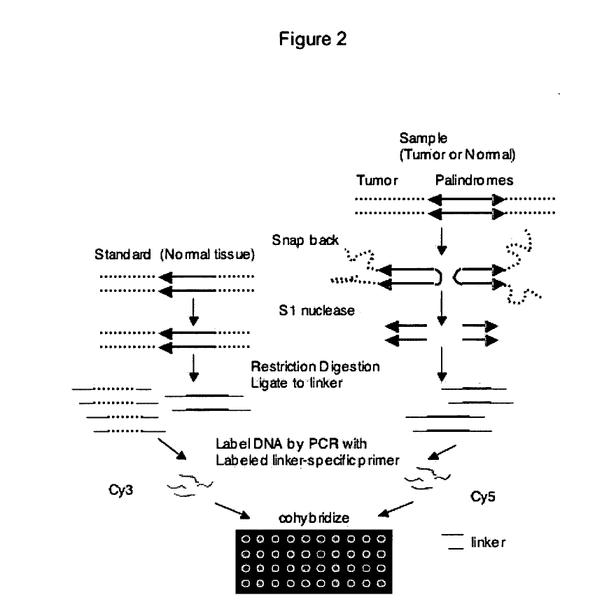

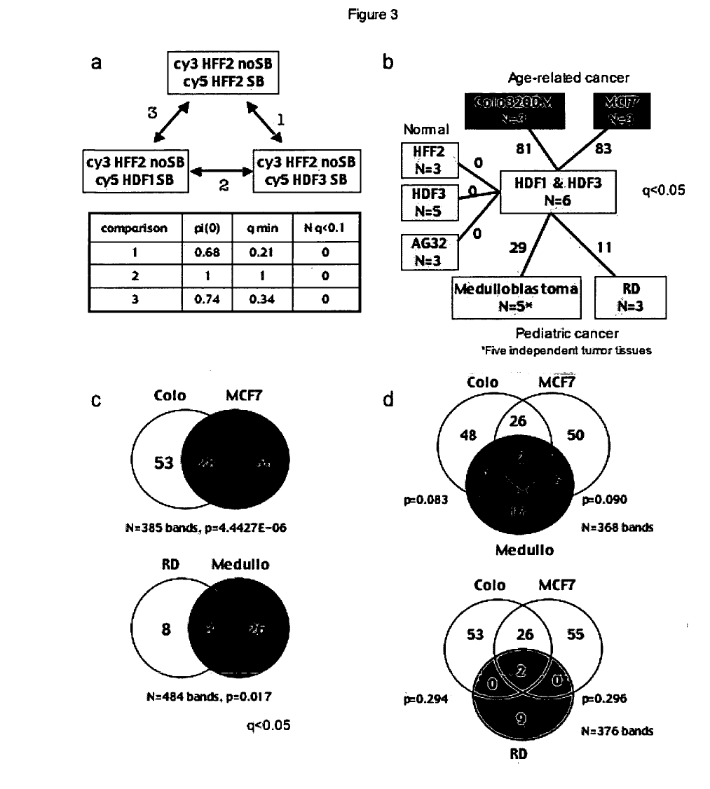

With this technique, genome-wide analysis of palindrome formation (GAPF) can be assessed using DNA array hybridization. Initially, genomic DNA was used from primary cultures of human fibroblasts derived from three different individuals (HDF1 (skin biopsy), HFF2 (foreskin sample) and HDF3 (skin biopsy)). It was assumed that somatic DNA palindrome formation was related to genetic instability and that normal fibroblasts would not have many differences between them. Genomic DNA from each of the fibroblasts was subjected to denaturation and rapid-renaturation (snap-back, or SB DNA); digested with S1 nuclease and restriction enzymes (MspI, TaqI or MseI); ligated to a linker specific for each enzyme; and amplified by PCR amplification with Cy-5 labeled linker specific primers (FIG. 2). For the common standard competitor DNA, genomic DNA was used from similarly processed HFF2 fibroblasts but without denaturation (non-SB DNA) and amplified using Cy-3 labeled linker specific primers. Cy-3 labeled non-SB HFF2 DNA was competitively hybridized against Cy-5 labeled SB DNA from HFF2, HDF1, or HDF3 on spotted arrays containing 18,000 (18 k) human cDNAs, generating comparable GAPF profiles of fibroblasts from each individual. For each fibroblast DNA, three independent preparations of SB DNA were processed for hybridization. The Storey's q-value, a measure of significance in terms of false discovery rate (FDR), was calculated for each gene in each comparison between fibroblasts to control for multiple testing errors. At a threshold of q<0.1, no features showed a significant difference between any two of the normal fibroblast samples (FIG. 3A).

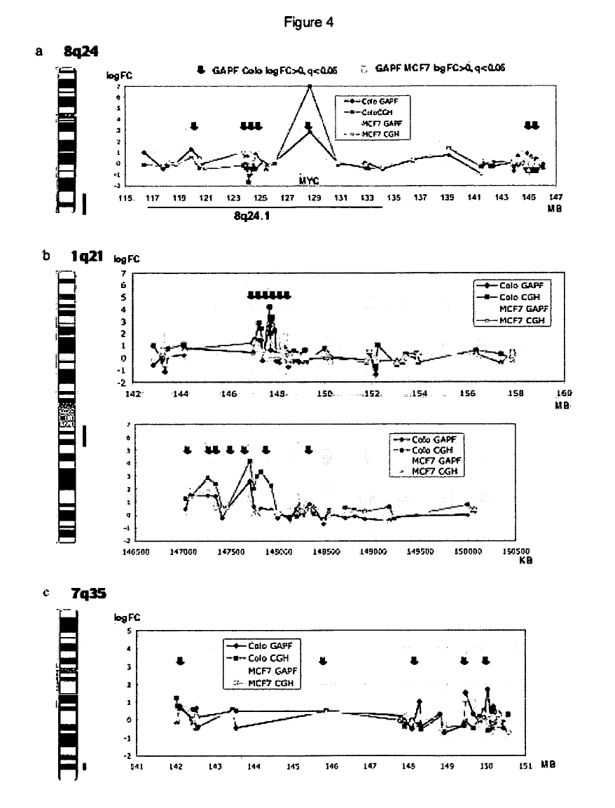

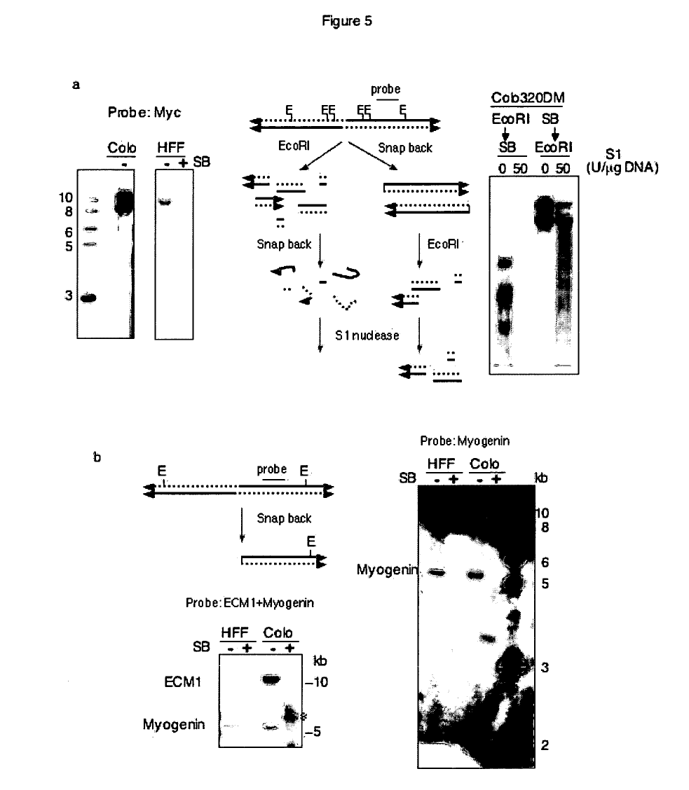

To determine whether GAPF can detect palindromes formed in cancer cells, the Colo320DM human colon cancer cell line (Colo) that has a large inverted repeat of the cMyc gene was used initially. SB DNA from Colo was labeled with Cy-5 and co-hybridized with the Cy-3 labeled non-SB DNA of HFF2. Experiments were performed in triplicate and the GAPF profile was compared to a ‘common baseline’ GAPF profile consisting of two triplicate data sets of SB DNA from the HDF1 and HDF3 fibroblasts (FIG. 3B). For this analysis, the data from individual genes was grouped into 521 cytogenetic bands that ranged in size from 1 to 132 genes with an average of 18 genes per cytogenetic band. Locating each gene on a physical map of cytogenetic bands helped to identify regions susceptible to palindrome formation. Based on a criteria of a q-value<0.05 and a log-fold change>0, there were no differences between the common baseline and the HFF2 GAPF, whereas 81 cytogenetic bands were increased in the Colo GAPF (FIG. 3B), indicating increased numbers of palindromes in the Colo DNA when compared to normal fibroblast DNA. As predicted, the cytogenetic band that includes cMyc, 8q24.1, showed a significant increase in Colo (q=0.024). This band covers 18 genes in a 13 Mb region and the increased features show a bimodal distribution: cMyc is GAPF-positive and there was also a cluster of three genes (ZHX2, MGC21654, and annexin A13) in a ˜900 kb region located 5 MB centromeric to cMyc that are also GAPF-positive (FIGS. 4A and 5A), which is consistent with a previous report that cMyc is amplified as a large inverted repeat in this cell line. A similar clustering of GAPF increased genes was also identified at 1q21 (FIG. 4B). This cytogenetic band was significantly increased in Colo (q=5.53×10⁻⁵), with three individual genes (Histone 2 (HIST2H2BE), vacuolar protein sorting 45A (VPS45A) and extracellular matrix protein 1 (EMC1), CKIP1 and FLJ23221) clustering within 600 kb (FIGS. 4B and 5B). Two additional genes (CK2 interacting protein 1 and FLJ23221) with a significant increase are also assigned to this region, indicating that this subregion of a cytogenetic band was a hotspot for a palindrome formation.

For comparison, a GAPF profile was obtained for a breast cancer cell line, MCF7, a normal breast epithelial cell line (AG 11132), and a rhabdomyosarcoma cell line, RD. No cytogenic bands were GAPF-positive in the comparison of AG11132 with the normal HDF fibroblast baseline, whereas eighty-three cytogenetic bands and 73 bins were significantly increased in MCF7 relative to the HDFs (FIG. 3B), including both 8q24.1 (q=0.035) and 1q21 (q=0.0056). At 8q24.1, the increased genes were the same four as are increased in the Colo cells (FIG. 5A). At 1q21, the increased genes include three that were also increased in Colo (Histone 2 (HIST2H2BE), Vacuolar protein sorting 45A (VPS45A) and Extracellular matrix protein 1 (ECM1)) (FIG. 4B). Overall, there was a significant overlap of the palindrome containing cytogenetic bands in Colo and MCF7 (28 bands, p=3.4427×10⁻⁶ and 20 bins, p=4×10⁻⁶) (FIG. 3C), indicating that these epithelial tumor cell lines from age-related cancers have common hotspots of palindrome formation. Similar to the analyses based on cytogenic bands or bins, there is also a significant overlap of GAPF-positive genes between Colo (150 genes) and MCF7 (388 genes) (40 genes in common, p<1×10⁻⁹⁹).

The GAPF profile of the RD cell line, derived from an embryonal rhabdomyosarcoma, identified 11 palindrome-containing cytogenetic bands. These 11 bands do not show significant overlap with those of Colo (p=0.29) or MCF7 (p=0.29), indicating that distinct GAPF patterns were associated with different types of tumor cells. It is interesting that the 2q35 band was identified as containing a palindrome in RD cells and the PAX3 gene in this region was enriched but did not meet the preset statistical criteria to be independently called elevated. Alveolar rhabdomyosarcomas are characterized by a t(2;13)(q35;q14) translocation that fuses the PAX3 gene with the FKHR gene on chromosome 13, whereas embryonal rhabdomosarcomas do not carry this translocation; however, the association of this region with a somatic palindrome formation in an embryonal rhabdomosarcoma indicates that PAX3 resides in a GAPF hotspot in this cell type and suggested that the alternative resolutions of a double-stranded break at this hotspot might determine the subtype of rhabdomyosarcoma generated.

Interestingly, the formation of palindromes at the GAPF hotspots was not always associated with an increase in gene copy number, as measured by comparative genomic hybridization (array-CGH). For example, at both 8q24.1 and 1q21, palindrome formation was associated with a significant increase (more than two-fold) in copy number in Colo but not in MCF7. In Colo, the cMyc associated palindrome at 8q24.1 was amplified, whereas the cluster of palindrome embedded genes in the adjacent region 5 MB centromeric to cMyc was not amplified. This discrepancy between the GAPF profile and array-based CGH indicates that the two approaches are measuring different features in the cancer cells: GAPF measures a structural feature (palindrome) and CGH measures the average copy number. In fact the majority of the genes that are significantly increased by GAPF in Colo were not identified as increased by CGH; however, GAPF genes were significantly more likely to be amplified than other loci, indicating that a subset of GAPF loci were selected for amplification. These data suggest that BFB cycles drive tumor progression by forming somatic palindromes at the specific loci, some of which are selected for gene amplification. For example, two of the three Colo loci (8q24.1 and 1q21) that include genes with more than a three-fold increase in copy number by CGH were associated with palindrome formations by GAPF. Also, the DUSP22 gene, another gene that shows more than three-fold amplification at 6p25 by array-CGH was associated with palindrome formation at the gene level, although 6p25 itself was not identified as a palindrome-containing cytogenetic band based on our predetermined statistical criteria. In contrast, at 7q35, where a common fragile site (FRA7I) is implicated as a chromosome break site in the palindromic amplification of the PIP oncogene in a breast cancer cell line, a gene (Contactin associated protein-like 2) has a palindrome formation in both Colo and MCF7 with a low-level increase in copy number in Colo, whereas two other genes (Zincfinger protein 289 and potassium voltage-gated channel, subfamily H) demonstrated palindromes in Colo with a low-level decrease in copy number. These data indicated that unstable hotspots in the cancer genome resulted in clustered areas of palindrome formation that serve as a platform for gene amplification.

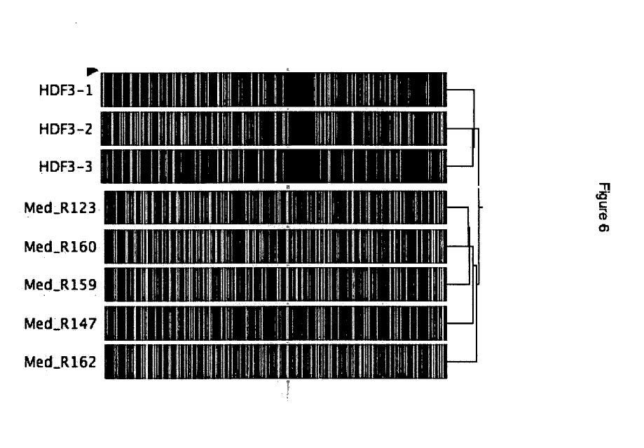

Colo, MCF7, and RD are cell lines derived from primary tumors and it is possible that the widespread palindrome formation revealed by GAPF might be secondary to multiple passages in culture. To examine somatic palindrome formation in primary tumors, GAPF analysis was performed on DNA isolated from five independent primary medulloblastomas, the most common central nervous system malignancy of childhood. Each tumor sample was processed as a singleton and the GAPF profiles from the five independent samples compared to the HDF GAPF profile. Somatic palindrome formation was detected at 29 cytogenetic bands in the primary human medulloblastomas (q<0.05) (FIG. 3B) and hierarchical clustering showed a high degree of similarity among individual medulloblastomas, which have a GAPF pattern that was clearly similar to each other and distinct from Colo and MCF7 (FIG. 6 and FIG. 3D). These palindrome-containing loci include 6q (6q12, 6q14), 4q (4q24, 4q25) and 7q (7q21.1, 7q22.1 and 7q31), which were commonly amplified in medulloblastoma tissues. Other GAPF-positive loci, such as 1p34.2, 5p15.2, 5p15.3 and 13q34, have been identified as highly amplified loci in a subset of medulloblastomas, suggesting a link between gene amplification and palindrome formation. The fact that five independent primary tumors have common loci of somatic palindrome formation indicates a shared mechanism of palindrome formation and indicated that tumor specific mechanisms determine their genomic location. It was interesting to note that the palindromic regions contained genes that likely contribute to tumor progression: Skp2 at 5p13 encodes a subunit of ubiquitin ligase complex that regulates entry into S phase by inducing the degradation of the cyclin dependent kinase inhibitors p21 and p27; Fzd1 at 7q21.1 encodes a receptor for the Wnt signaling pathway that is often dysregulated in medulloblastomas; and, Tert, telomere reverse transcriptase at 5p15.3 is often amplified in medulloblastomas.

In contrast to the similarity of the Colo and MCF7 GAPF profiles, there was no significant overlap of cytogenetic bands between medulloblastomas and Colo320DM (p=0.08) or between medulloblastomas and MCF7 (p=0.09); however, significant overlap was evident between medulloblastomas and RD (p=0.01) (FIG. 3C), despite the much smaller number of palindrome containing cytogenetic bands in RD. These results indicated a different distribution of somatic palindromes in pediatric tumors (medulloblastomas and rhabdomyosarcomas) and age-related cancers (colon and breast), suggesting that the mechanisms responsible for palindrome formation at specific loci might reflect fundamental properties of tumor cell biology.

Discussion

These results identify widespread somatic palindromes that occur in characteristic patterns in specific cancer types. Unlike conventional array-CGH (comparative genomic hybridization) analysis that measures the average gene dosage in cell populations, GAPF provides a qualitative measurement of a structural chromosomal aberration (palindromes) that has previously been examined only by cytogenetic studies. Detailed mapping of the palindromes on the physical genome reveals that palindrome formations tend to cluster at specific regions, some of which undergo gene amplification. In addition, the pattern of genome wide palindrome formation appears to be different among different types of cancers, indicating that the palindrome formation reflects specific differences in the biology of each cancer type.

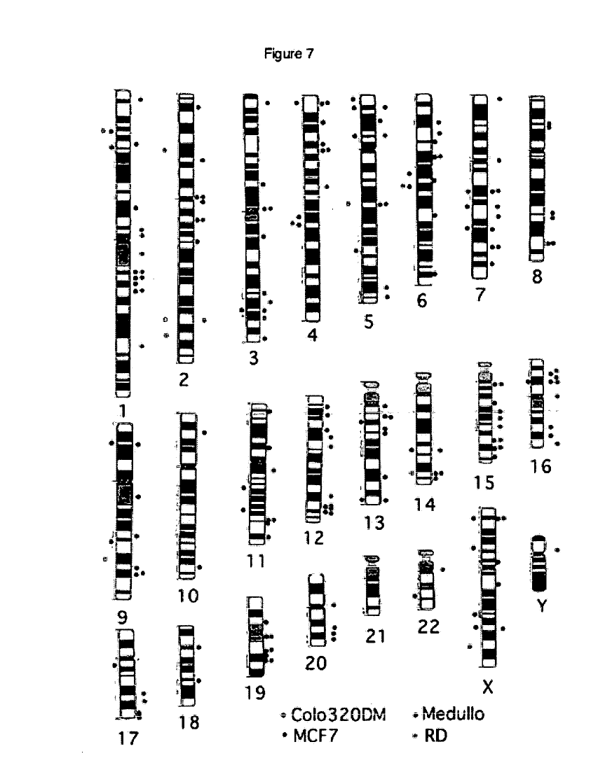

The clustering of somatic palindromes could be due to clustering of chromosome breakage sites in the genome, since chromosome breakage is required for palindrome formation. Cytogenetic studies have shown that clastogenic drug-induced fragile sites are involved in inverted duplications and gene amplifications in rodent cells (Coquelle et al., Cell 89:215-225 (1997)), and aphidicolin-induced fragile sites are involved in oncogene amplification in human cancer cells (Ciullo et al. Hum. Mol. Genet. 11:2887-2894 (2002); Hellman et al., Cancer Cell 1:89-97 (2002)). In fact, the GAPF-positive cytogenetic bands detected in both the Colo320DM human colon cancer cell line and the MCF7 breast cancer cell line were co-localized at 1q21, 8q24.1, 12q24, 16p12-13.1 and 19q13, which all harbor common fragile sites (FIG. 7). Although the majority of the common fragile sites remain to be characterized at the molecular level, the fact that palindromes cluster at these loci suggests a role for common fragile sites in palindrome formation. Stability of common fragile sites is controlled, in part, by the replication checkpoint kinase ATR (Casper et al., Cell 111:779-789 (2002)). In yeast, impaired function of the ATR homologue Mce1 leads to stalled replication forks and chromosome breaks in specific regions of the genome (Cha and Kleckner, Science 297:602-606 (2002) that can result in gross chromosome rearrangement (Myung et al., Cell 104:397-408 (2001)). Compromised checkpoint function might generate similar chromosome breaks and somatic palindromes in specific regions of the genome in cancer cells. In addition to common fragile sites, topoisomerase cleavage sites might determine sites of initial DNA double strand breakage, which have been shown to initiate disease-associated chromosomal translocations (Domer et al., Proc. Natl. Acad. Sci. USA 90:7884-7888 (1993); Dong et al., Genes Chrom. Cancer 6:133-139 (1993); Hirai et al., Genes Chrom. Cancer 26:92-96 (1999); Lovett et al., Proc. Natl. Acad. Sci. USA 98:9802-9807 (2001); Obata et al., Genes Chrom. Cancer 26:6-15 (1999)). It is also interesting that a number of GAPF positive genes are associated with translocations in some tumor types, such as T-cell leukemia/lymphoma 1A (TCL1A) (Davey et al., Proc. Natl. Acad. Sci. USA 85:9287-9291 (1998); Erickson et al., Science 229:784-786 (1985); Hecht et al., Science 226:1445-1447 (1984)); Synovial sarcoma, X-breakpoint 4 (SSX4) (Skytting et al., J. Natl. Cancer Inst. 91:974-975 (1999), and Myeloid leukemia factor 1 (MLF1) (Yoneda-Kato et al., Oncogene 12:265-275 (1996)). Therefore, it is possible that chromosome breaks at these genes might be resolved either as a palindrome or as a translocation with significantly different consequences to the progression of the tumor.

In RD, 2q35 was identified as GAPF-positive and the PAX3 gene in this region was enriched by GAPF, although did not meeting the present statistical criteria to be independently call elevated as a single gene. Alveolar rhabdomyosarcomas are characterized by a t(2;13)(q35;q14) translocation that fuses the PAX3 with the FKHR gene on chromosome 13, whereas embryonal rhabdomyosarcomas do not carry this translocation (Anderson et al. Genes Chrom. Cancer 26:275-285 (1999)); however, the association of this region with a somatic palindrome formation in an embryonal rhabdomyosarcomas indicates that PAX3 resides in a GAPF hotspot in this cell type and suggests that the alternative resolutions of a double-stranded break at this hotspot might determine the subtype of rhabdomyosarcoma generated. For medulloblastoma, it is also interesting to note that the palindromic regions contain genes that might contribute to tumor progression: Skp2 at 5p13 encodes a subunit of ubiquitin ligase complex that regulates entry into S phase by inducing the degradation of the cyclin dependent kinase inhibitors p27 (Carron et al., Nat. Cell Biol. 1:193-199 (1999)); Fzd1 at 7q21.1 encodes a receptor for Wnt signaling pathway that is often dysregulated in medulloblastomas (Yokota et al., Int. J. Cancer 101:198-201 (2002)); and Tert, telomere reverse transcriptase at 5p15.3 is often amplified in medulloblastomas (Fan et al., Am. J. Pathol. 162:1763-1769 (2003)).

In addition to the requirement for a double-strand break, other cis-acting sequences might determine where palindromes can form. In the simple eukaryotes Tetrahymena (Butler et al., Mol. Cell. Biol. 15:7117-7126 (1995); Yao et al., Cell 63:763-772 (1990); Yasuda and Yao, Cell 67:505-516 (1991)), yeast, e.g., S. pombe (Albrecht et al., Mol. Biol. Cell 11:8730886 (2000)), and Leshmania (Grondin et al. Mol. Cell. Biol. 16:3587-3595 (1996)), palindrome formation is mediated by a pair of short inverted repeats that naturally exist in the genome. In S. cervisiae, exogenous short inverted repeats consisting of human Alu repeats inserted in the chromosome can induce chromosome breaks and palindrome formation in an Mre11 mutant background (Lobachev et al., Cell 108:183-193 (2002)). In CHO cells, we have directly shown that short inverted repeats can mediate palindrome formation following an adjacent double-strand break, which leads to subsequent BFB cycles and gene amplification (Tanaka et al., Proc. Natl. Acad. Sci. USA 99:8772-8777 (2002)). Short inverted repeats are common in the human genome and are often involved in disease-related DNA rearrangements (Kurahashi and Emanuel, Hum. Mol. Genet. 10:2605-2617 (2002); Kurahashi et al., Am. J. Hum. Genet. 72:733-738 (2003)). Further studies might determine whether naturally occurring short inverted repeats facilitate the widespread palindrome formation we have characterized in cancer cells.

Alveolar rhabdomyosarcomas are characterized by a t(2;13)(q35;q14) translocation that fuses the PAX3 and FOXO1A genes on chromosome 13, whereas embryonal rhabdomyosarcomas do not carry this translocation; however, the association of this region with a somatic palindrome formation in an embryonal rhabdomyosarcoma RD implies that PAX3 also resides in a region susceptible to DSBs and suggests that the alternative resolutions of a DSB might determine the subtype of rhabdomyosarcoma generated.

Surprisingly, most of the loci with palindromes are not associated with an increase in gene copy number. In addition, the cancer cells from age-related epithelial cancers form palindromes at similar locations, whereas five different primary medulloblastomas have their own distinct pattern of palindrome distribution, which is similar to a pediatric rhadomyosarcoma derived cancer cell line. It appears, therefore, that sets of cancer types share common profiles of palindrome formation. Subsequent gene amplification might occur at subsets of these loci given tumor-specific selective pressure for growth. For example, palindromes cluster at 1q21 and 8q24 in both Colo320DM and MCF7, however, copy number is increased only in Colo320DM. This indicates that palindrome formation might be an early and fundamental step in cancer formation, providing a platform for subsequent gene amplification at a restricted set of loci. In this model, different tumor types might have a common set of palindromes, but the selective advantage of a given locus would determine its subsequent amplification in the cancer. The identification of widespread palindrome formations specific to different types of cancers provides a new opportunity to develop sensitive assays for detection of residual disease, early detection, and tumor classification. Ultimately, preventing the underlying mechanisms that lead to widespread palindrome formation might prevent tumor initiation.

### Example 2

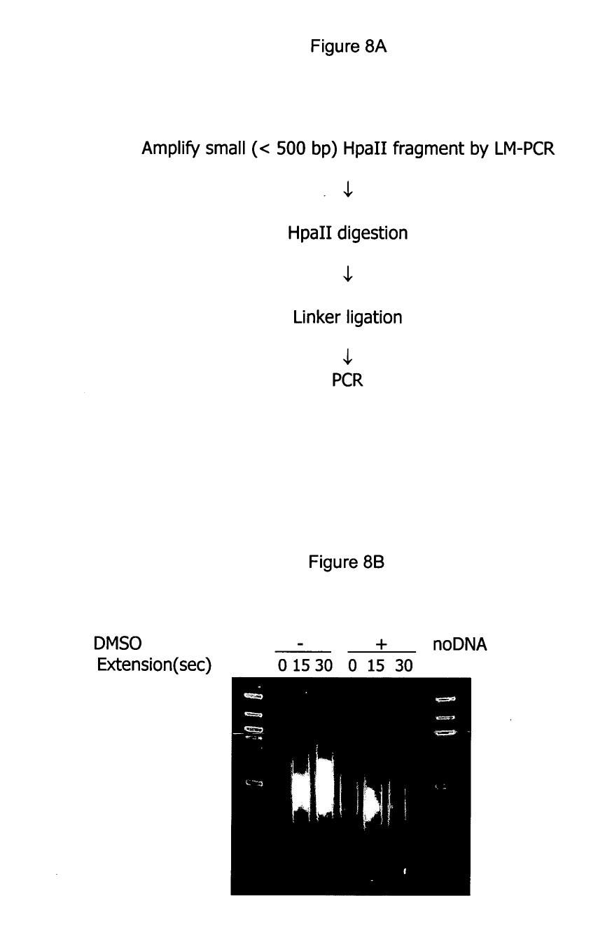

The following example demonstrates the use of ligation-mediated PCR to isolated a DNA fragment enriched in unmethylated CpG islands in a mammalian cell. A schematic of the process is provided as FIG. 8A. The methods for

Briefly, mouse genomic DNA was digested with a methylation sensitive restriction enzyme (for example, HpaII). The MspI linkers used above in Example 1 were used to ligate the HpaII fragments. The ligated DNA was amplified by PCR using the MspI primer from Example 1 (SEQ ID NO: 6). The method resulted in the specific amplification of HpaII digested genomic DNA of less than 500 base pairs. (FIG. 8B). Random cloning and sequencing of the PCR products revealed that more than 50% of clones were at the CpG islands as defined using stringent criteria. (Takai and Jones, Proc. Natl. Acad. Sci USA 99:3740-3745 (2002); incorporated herein by reference). In contrast, amplification of DNA digested with methylation-resistant isoschizomer MspI gave no clones near CpG islands.

TABLE 1
Results of random sequencing.

| | n | GC content | CpG Island |
|---|---|---|---|
| HpaII | 20 | 56.2% | 11 (55%) |
| | | (43-68%) | |
| MspI | 11 | 50.6% | 0 (0%) |
| | | (43-59%) | |

A systematic study of the methylation status of CpG islands throughout the genome becomes possible by combining this approach with human or mouse CpG island microarrays. For example, the labeled unmethylated DNA fragments can use to interrogate a microarray DNA library constructed from a particular organism or tissue from a particular organism. The result with this library can be compared to a DNA library constructed from a different tissue or the same tissue from a different developmental period. The differences between the methylation patter determined from each tissue sample can indicate changes in DNA methylation associate with, for example, tumorigenesis, or development.

The previous examples are provided to illustrate but not limit the scope of the claimed inventions. Other variations of the inventions will be readily apparent to those of ordinary skill in the art and encompassed by the following claims. All publications, patents and patent applications and other references cited herein are hereby incorporated by reference.

What is claimed is:

1. A method for identifying a region of genomic DNA comprising a DNA palindrome, comprising incubating isolated genomic DNA under conditions conducive to snap back DNA formation and not inter-molecular hybridization, the snap back DNA containing the DNA palindrome; isolating the snap back DNA; and identifying the regions of the genomic DNA comprising the snap back DNA thereby identifying the region of the genomic DNA comprising the DNA palindrome.

2. The method according to claim 1, wherein the method comprises: fragmenting the genomic DNA, denaturing the genomic DNA, incubating the fragmented, denatured genomic DNA under conditions conducive to the formation of snap back DNA by regions of the DNA comprising the DNA palindrome; and identifying the region of the genomic DNA containing the DNA palindrome by hybridization with a human genomic DNA array.

3. The method according to claim 2, wherein the method comprises the steps of:
a) isolating genomic DNA comprising the DNA palindrome from a population of cells;
b) denaturing the isolated DNA;
c) rehybridizing the denatured isolated DNA under suitable conditions for the DNA palindrome to form snap back DNA;
d) digesting the rehybridized DNA with a nuclease that digests single stand DNA to form double stranded DNA fragments comprising the snap back DNA;
e) digesting the double stranded DNA fragments comprising the snap back DNA with a nucleotide sequence specific restriction enzyme;
f) adding a sequence specific linker nucleotide sequence to one end of each stand of the double stand DNA comprising the snap back DNA;
g) amplifying the DNA fragments comprising the added linker using a labeled linker sequence specific primer corresponding to the sequence specific linker added in step (f);
h) hybridizing the amplified DNA fragments comprising the snap back DNA to a genomic DNA library and identifying the genomic DNA region comprising the palindrome.

4. The method according to claim 3, wherein the amplified DNA fragments comprising the snap back DNA are mixed and co-hybridized in step (h) with a sample of high molecular weight DNA from a normal cell population that has been digested with S1 nuclease, and the restriction enzyme of step (e), adding a linker labeled with a second single label, and amplified.

5. The method according to claim 3, wherein the single strand nuclease comprises S1 nuclease.

6. The method according to claim 3, wherein the restriction enzyme comprises MspI, TaqI, or MseI.

7. The method according to claim 3, wherein the genomic DNA is fragmented by a chemical, physical, or enzymatic method.

8. A method for classifying a population of cancer cells, comprising identifying a plurality of snap back DNA regions that comprise genomic DNA regions containing a palindrome and using the identity of the plurality of genomic DNA regions comprising the palindromes to classify the population of cancer cells.

9. The method according to claim 8, wherein the method of identifying the plurality of genomic DNA regions comprising a palindrome comprises fragmenting the genomic DNA; denaturing the genomic DNA; incubating the fragmented, denatured genomic DNA under conditions conducive to the formation of snap back DNA by regions of the DNA comprising the DNA palindrome; and identifying the region of the genomic DNA containing the DNA palindrome to form the profile.

10. The method of claim 9, further comprising comparing the profile of genomic DNA comprising a palindrome of the cancer cell population to a population of normal cells.

11. A method for detecting a population of cancer cells, comprising isolating genomic DNA from a cell population, identifying a plurality of snap back DNA regions that comprise genomic DNA regions containing a palindrome and using the identity of the plurality of genomic DNA regions comprising the palindromes to detect the population of cancer cells.

12. The method according to claim 11, wherein the method of identifying the plurality of genomic DNA regions comprising a palindrome comprises fragmenting the genomic DNA; denaturing the genomic DNA; incubating the fragmented, denatured genomic DNA under conditions conducive to the formation of snap back DNA by regions of the DNA comprising the DNA palindrome; and identifying the region of the genomic DNA containing the DNA palindrome to form the profile.

13. The method of claim 12, further comprising comparing the profile of genomic DNA comprising a palindrome of the cancer cell population to a population of normal cells.

14. A method for determining a region of genomic DNA that comprises a unmethylated CpG island, comprising:
a) digesting genomic DNA with a methylation sensitive restriction enzyme;
b) amplifying the DNA fragments using a labeled linker sequence;
c) hybridizing the amplified DNA fragments to a genomic DNA library and identifying the genomic DNA region comprising the palindrome.
